(19) United States
(12) Patent Application Publication
(10) Pub. No.: US 2025/0082412 A1
(43) Pub. Date: Mar. 13, 2025

(54) OPHTHALMIC SURGICAL ROBOT

(71) Applicant: Alcon Inc., Fribourg (CH)

(72) Inventors: Patrick TERRY, Goleta, CA (US); Mark SMYTHE, Fort Worth, TX (US); Sissimos LEMONIS, Rückersdorf (DE); Satish YALAMANCHILI, Lake Forest, CA (US); Russell FINLAY, Keller, TX (US); Nanhong LOU, Southlake, TX (US)

(21) Appl. No.: 18/830,304

(22) Filed: Sep. 10, 2024

Related U.S. Application Data
(60) Provisional application No. 63/582,227, filed on Sep. 12, 2023.

(51) Int. Cl. A61B 34/20; A61B 34/30

(52) U.S. Cl. CPC A61B 34/20; A61B 2034/306

(57) ABSTRACT

A system for performing ophthalmic surgery includes a robotic positioning system including an end effector. The robotic positioning system is configured to position the end effector with at least five degrees of freedom. An ophthalmic surgical instrument is mounted to the end effector along with an accessory device configured to facilitate performance of an ophthalmic treatment by the ophthalmic surgical instrument. The robotic positioning system may be controlled to enforce anatomical boundaries and implement predefined motion profiles.

### CROSS-REFERENCE TO RELATED APPLICATIONS

This application claims priority to U.S. Provisional Application No. 63/582,227, filed on Sep. 12, 2023, which is hereby incorporated by reference in its entirety.

### BACKGROUND

The present disclosure relates generally to performing ophthalmic surgery.

Light received by the eye is focused by the cornea and lens of the eye onto the retina at the back of the eye, which includes the light sensitive cells. The area between the cornea and the lens is known as the anterior chamber. The interior of the eye between the lens and the retina is known as the posterior chamber and is filled with a transparent gel known as the vitreous. Many ocular pathologies may be treated by performing ophthalmic treatments in the interior or posterior chamber.

It would be an advancement in the art to facilitate the performance of ophthalmic treatments.

### SUMMARY

In certain embodiments, a system for performing ophthalmic surgery includes a robotic positioning system including an end effector, the robotic positioning system configured to position the end effector with at least five degrees of freedom. An ophthalmic surgical instrument is mounted to the end effector. An accessory device is mounted to the end effector and configured to facilitate performance of an ophthalmic treatment by the ophthalmic surgical instrument.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A is an isometric view of a robotic arm having a surgical instrument and an imaging device mounted thereto in accordance with certain embodiments.

FIG. 1B is a diagram illustrating the implementation of a remote center of motion in accordance with certain embodiments.

FIG. 1C is a plot of points within the posterior chamber of an eye in accordance with certain embodiments.

FIG. 2A is a diagram showing an ophthalmic treatment performed using the surgical instrument and imaging device in accordance with certain embodiments.

FIG. 2B is a diagram showing an ophthalmic treatment performed using a surgical instrument incorporating a laser and an imaging device in accordance with certain embodiments.

FIG. 2C is a diagram showing an ophthalmic treatment performed using a surgical instrument incorporating a treatment laser and an imaging device in accordance with certain embodiments.

FIG. 2D is a diagram showing an ophthalmic treatment performed using a surgical instrument including forceps and an imaging device in accordance with certain embodiments.

FIG. 3 is a diagram illustrating a teleoperated robotic arm in accordance with certain embodiments.

FIG. 4 is a process flow diagram of a method for controlling the teleoperated robotic arm in accordance with certain embodiments.

FIG. 5 is a diagram illustrating a robotic arm and external light source in accordance with certain embodiments.

FIG. 6 is a diagram illustrating a robotic arm and a light source inserted within the posterior chamber of an eye in accordance with certain embodiment.

FIGS. 7A and 7B illustrate example probes including one or more light sources in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

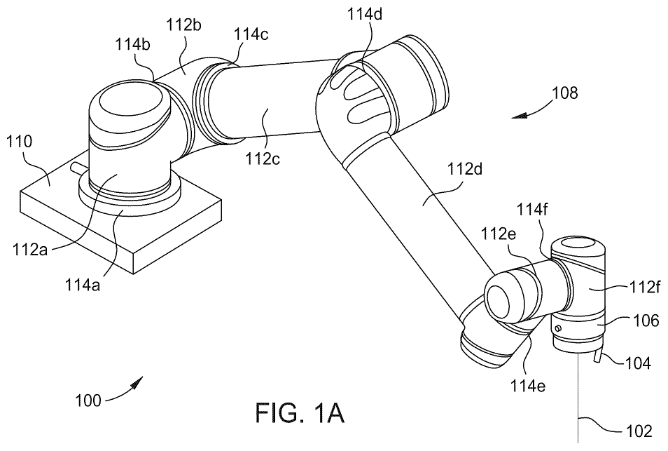

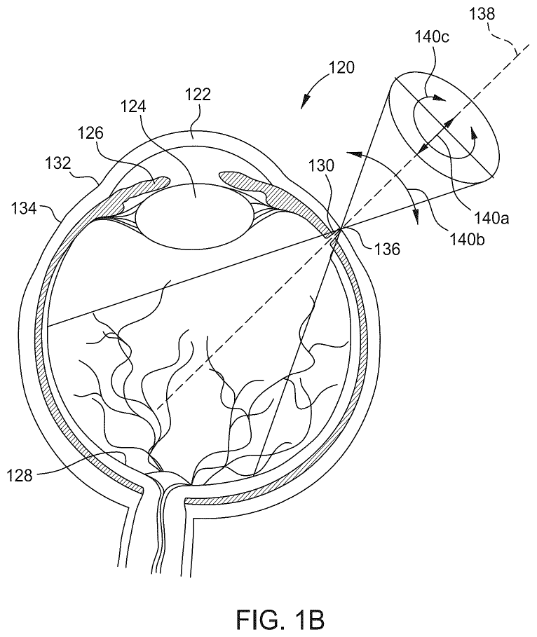

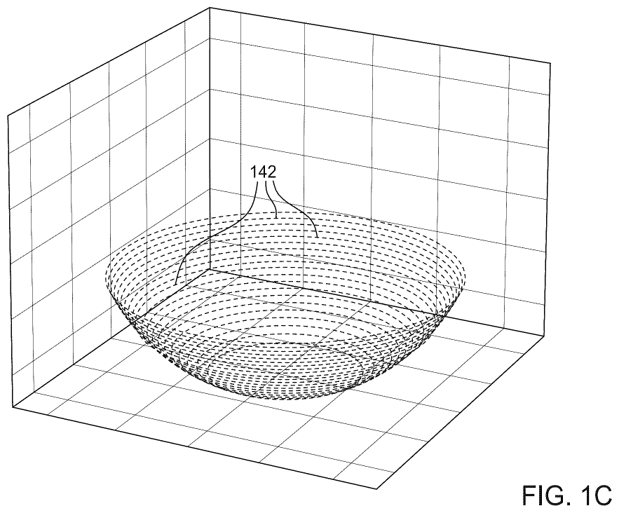

Referring to FIG. 1A, system 100 for performing an ophthalmic treatment or assisting in the performance of an ophthalmic treatment by a surgeon includes an instrument 102 configure to interact with tissues of an eye of a patient. The system 100 further includes an accessory device 104. The accessory device 104 may include some or all of a light source, a treatment laser, a two- or three-dimensional surface imaging device (e.g., monoscopic or stereoscopic camera), a sectioning imaging device (e.g., optical coherence tomography imaging device (OCT)), or other type of imaging device. Example accessory devices 104 and the uses thereof are described in greater detail below.

The instrument 102 and accessory device 104 may function as, or be connected to, the end effector 106 of a robotic arm 108. The robotic arm 108 includes a base 110 and a plurality of links 112a-112f coupled to the base 110, one another, and the end effector 106 by one or more joints 114a-114f. The base 110 may be fixed relative to a support surface, such as by mounting to a floor, ceiling, wall, or mobile cart having the wheels thereof immobilized. Collectively the joints 114a-114f define one or more degrees of freedom, such as at least 5 degrees of freedom or at least 6 degrees of freedom. The degrees of freedom enable the robotic arm 108 to position the end effector 106 within two- or three-dimensional space and orient the end effector with two or three degrees of rotational freedom. A kinematic solution by which a position and orientation of the end effector 106 is translated into orientations of the links 112a-112f may be computed according to any approach known in the art.

The robotic arm 108 is one example of the robotic positioning system that may be used to position an end effector 106 as described herein. The robotic arm 108 may be substituted with other types of robotic positioning systems such as one or more linear rails and corresponding actuators (e.g., a multi-dimensional gantry), parallel actuation approaches, or other type of robotic actuator.

Referring to FIG. 1B, for example, for the eye 120 of a patient includes the cornea 122 and lens 124, that cooperate to focus light onto the light-sensitive cells of the retina 128. The space between the cornea 122 and lens 124 and iris 126 is known as the anterior chamber whereas the space between the lens 124 and the retina 128 is known as the posterior chamber and is filled with a transparent gel known as the vitreous. The instrument 102 may be inserted into the anterior or posterior chamber through an incision 130. For example, the incision may be at or near the limbus 132, which as the boundary between the cornea 122 and the sclera 134, i.e., the white of the eye. The incision 130 may also be in the sclera 134 itself as shown in FIG. 1B.

The robotic arm 108 may be controlled to maintain a remote center of motion (RCM) 136 at the incision 130, such as using a lock-to-target algorithm. For example, the lock-to-target algorithm may include the approach described in U.S. Pat. No. 11,336,804B2, which is hereby incorporated herein by reference in its entirety. The proximal end of the instrument 102 (external to the eye 120) may be moved by the robotic arm 108 in various directions defined with respect to an axis 138 of the incision 130, such as a trocar cannula placed in the incision 130. For example, an axial direction 140a may be defined as movement parallel to the axis 138, an angular direction 140b may be defined as rotation in a plane passing through the axis 138, and a precession direction 140c, defined as rotation about the axis 138. The position and orientation of the robotic arm 108 is independent of the position of a surgical microscope used by a surgeon to view the eye 120. Accordingly, a desired insertion location and orientation may be selected without requiring the surgical microscope to be placed at an angle that is awkward to the patient and/or surgeon. A surgical microscope may be implemented as the NGENUITY 3D VISUALIZATION SYSTEM provided by Alcon Inc. of Fort Worth Texas.

Referring to FIG. 1C, with freedom of movement about the directions 140a, 140b, 140c, the distal end of the instrument 102 may be positioned at an array of points 142 in three-dimensional space within the posterior or anterior chambers of the eye, including the illustrated spherical shape corresponding to the shape of the retina 128.

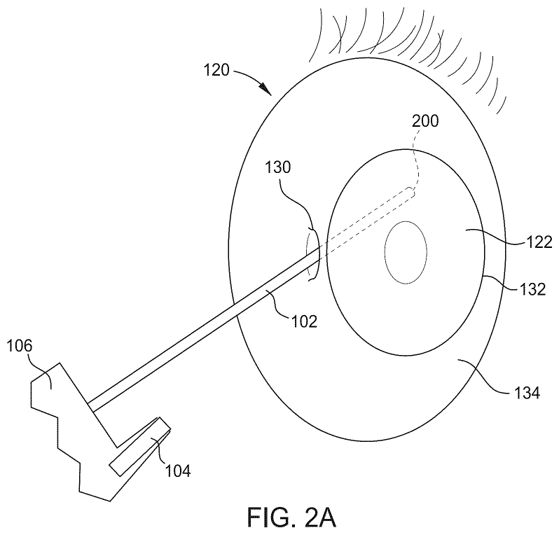

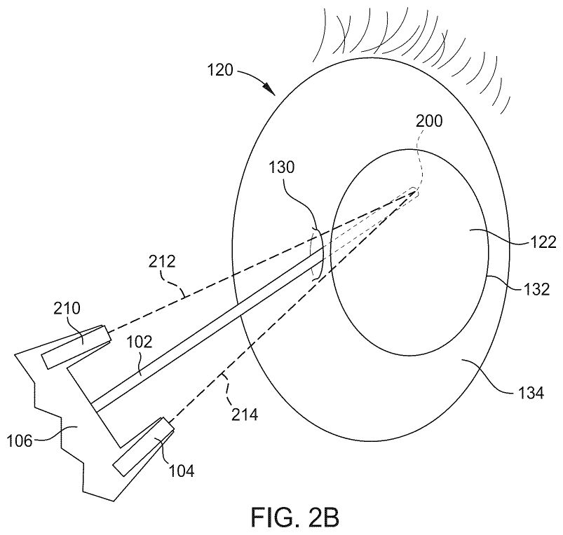

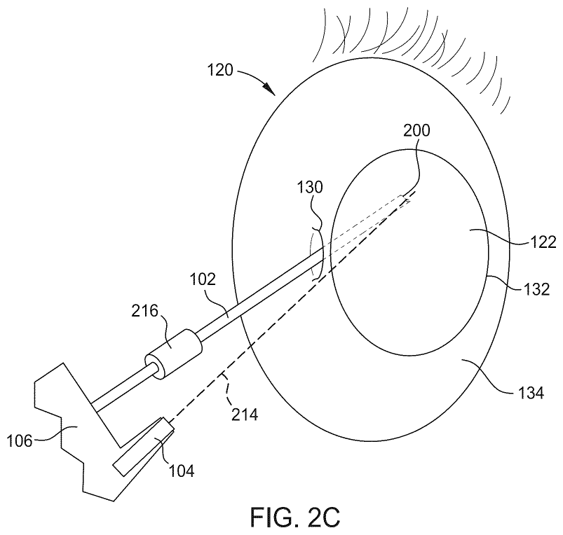

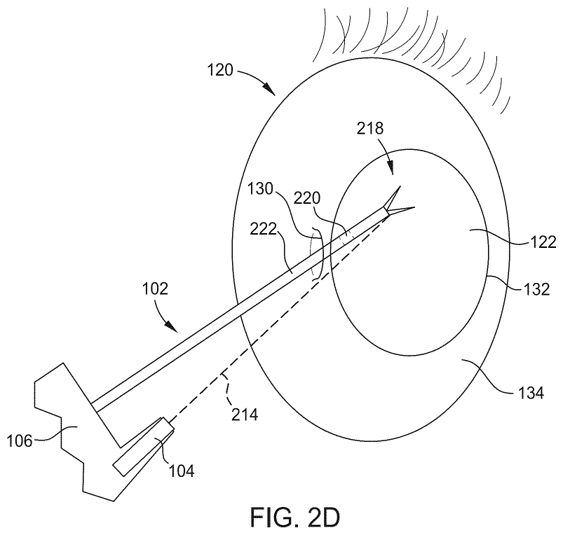

Referring to FIG. 2A, in some embodiments, the instrument 102 and accessory device 104 are used to perform an ophthalmic treatment on an eye 120. The treatment may be performed within the anterior chamber or the posterior chamber. For example, treatments to the anterior chamber may include treatments for glaucoma (e.g., minimally invasive glaucoma surgery (MIGS)), such as placing a stent or making an incision in the trabecular meshwork. The treatments may be for cataracts, such as performing rhexis, capsulotomy, and phacoemulsification followed by placement of an intraocular lens (IOL). Treatments to the posterior chamber may include a vitrectomy (removal of the vitreous), an intravitreal injection, peeling of a membrane (e.g., internal limiting membrane (ILM)) or epiretinal membrane (ERM), retina attachment, or other procedures. The instrument 102 and accessory device 104 may be used as part as a small incision lenticular extraction (SMILE) procedure.

The instrument 102 may therefore be embodied as an instrument for performing any of the above-reference procedures, including a phaco-vit tool for performing phacoemulsification or vitrectomy, an instrument for creating an incision or placing a stent, rod including a fiber optic cable for emitting laser pulses, forceps or other tool for performing membrane peeling, or other ophthalmic surgical instrument. The instrument 102 may be removably attached to the end effector 106 such that any number of different instruments 102 configured to perform any of the above-described ophthalmic treatments may be secured to the end effector 106.

The instrument 102 may be inserted into an incision 130. The incision 130 may be placed at or near the limbus 132, sclera 134, or elsewhere on the eye 120 and the instrument 102 inserted therethrough. During use of the instrument 102, the accessory device 104 may be used to facilitate safe performance of the ophthalmic treatment. In a first example, the instrument 102 is a phaco-vit instrument used to perform vitrectomy and the accessory device 104 is an OCT. The OCT may be used in various ways. Without the instrument 102 in place, the robotic arm 108 (see FIG. 1A) may move the OCT to various positions around the eye 120. Using a known location of the OCT from the kinematic state of the robotic arm 108, OCT section images from various locations may be used to generate a three-dimensional image of the eye 120. With the instrument 102 in place and during performance of an ophthalmic treatment, the OCT may continue be used to generate three-dimensional images of the eye 120, which may capture, for example, changes to the anatomy of the eye 120 resulting from the ophthalmic treatment and possibly the position of the distal end 200 of the instrument 102. The three-dimensional image may be generated using outputs of the OCT as well as one other imaging devices, such as images from a surgical microscope or other camera. For example, eye tracking may be performed using a camera. The three-dimensional image generate according to the output of the OCT may then be rotated in correspondence with movement of the eye detected by the eye tracking and aligned with images obtained using the surgical microscope or other camera.

For example, the three-dimensional image of the eye generated intraoperatively may be analyzed to identify anatomy of the eye 120 and the identified anatomy may be used in some or all of the following ways:
The three-dimensional images and possibly labels of anatomy identified therein may be displayed to the surgeon during the ophthalmic treatment in order to provide visualization of areas not visible using the surgical microscope.
The labels of anatomy may be used to control the robotic arm 108 or guide a surgeon. For example, labels of anatomy may be used in combination with a treatment plan provide precise locations of the anatomy of the eye 120 and the distal end 200 of the instrument 102 in order to guide the selection of locations to which to move the distal end 200 relative to the anatomy of the eye in order to perform an ophthalmic treatment, such as points of the posterior chamber from which to remove the vitreous, points within the capsular bag of the eye 120 at which to perform phacoemulsification, points within the trabecular meshwork to place incisions or stents, or locations in the retina at which to direct laser pulses for retinal attachment, or other actions.
Provide feedback to the surgeon or algorithm controlling the robotic arm 108 when the distal end 200 of the instrument 102 approaches a boundary, such as the retina, the capsular bag enclosing the lens 124, or other boundary defined in a treatment plan for the eye 120 of the patient.
Identify the amount remaining of material to be removed (membrane, vitreous, lens 204, etc.) in the three-dimensional map and provide feedback to the surgeon or an algorithm controlling the robotic arm 108 regarding where to move the distal end 200 of the instrument 102 in order to remove remaining material.

The above-listed examples are exemplary only and any other ophthalmic treatment may likewise be guided using the three-dimensional map, labels of anatomy of the eye in the three-dimensional map, and other data describing the ophthalmic treatment.

Referring to FIG. 2B, in another example use case, the accessory device 104 is an OCT and a treatment laser 210 is also coupled to the end effector 106 of the robotic arm 108. The focal point of the treatment laser 210 may be either (a) fixed at a point near (e.g., within 0.1 mm) the distal end of the instrument 102 and (b) variable with respect to parameters such as angle of the optical axis 212 of the treatment laser 210 and distance along the optical axis 212. Where variable, actuators mounting the treatment laser 210 to the end effector 106 may be used to vary the above-mentioned parameters of the focal point.

The treatment laser 210 may be used to perform actions such as photocoagulation, retinal attachment, making incisions (e.g., rhexis, capsulotomy, etc.) or other operations. The treatment laser 210 may be used in cooperation with the instrument 102, such as performing any of the above-described actions following a preceding action performed by the instrument 102, such as performing photocoagulation, retinal attachment or other operation following performance of a vitrectomy, peeling of a retinal membrane, or other action performed with respect to the retina 128 or elsewhere in the posterior chamber of the eye 120.

In the embodiment of FIG. 2B an optical axis 214 of the OCT may be fixed or controlled to substantially (e.g., within 0.1 mm) intersect the focal point of the treatment laser 210 in order to image the anatomy of the eye 120 to be treated by the treatment laser 210.

Referring to FIG. 2C, in yet another embodiment, the instrument 102 is itself part of a treatment laser system. The instrument 102 may include a treatment laser 216 and the instrument 102 may include an optical fiber conducting light from the treatment laser 216 to the distal end 200 of the instrument 102. Alternatively, the treatment laser 216 may be remote from the end effector 106 and instrument 102 with light likewise being conducted to the distal end 200 of the instrument 102 by an optical fiber. Light from the treatment laser 216 may be used to create incisions in the trabecular meshwork, perform photocoagulation, perform retinal attachment, or other actions.

As for the embodiment of FIG. 2B, the optical axis 214 of the OCT may be fixed or controlled to substantially (e.g., within 0.1 mm) intersect the focal point of the treatment laser 216 in order to image the anatomy of the eye 120 to be treated by the treatment laser 226.

Referring to FIG. 2D, in some embodiments, the instrument 102 includes forceps 218 at the distal end 200. The forceps 218 may be forceps configured to grasp a retinal membrane, such as the ILM or an ERM. The forceps 218 may be configured for placing sutures, such as corneal sutures, sutures for closing an incision 130, or other types of sutures. The forceps 218 may be configured to grasp and remove a lenticle as part of a SMILE procedure. A force/torque sensor 220 may be incorporated into the instrument 102, such as into a rod 222 to which the forceps 218 are mounted. The force/torque sensor 220 may additionally or alternatively be incorporated into one or both arms of the forceps 218. The force/torque sensor 220 measures the amount of force and/or torque transmitted through the forceps 218. The force/torque sensor 220 may be positioned at various locations on the instrument 102 other than the rod 222, such as a portion of the instrument 102 or rod 222 that is not inserted into the eye during use. A model of the instrument 102 may then be used to translate force/torque detected by the force/torque sensor 220 to force/torque exerted at the distal end of the rod 222.

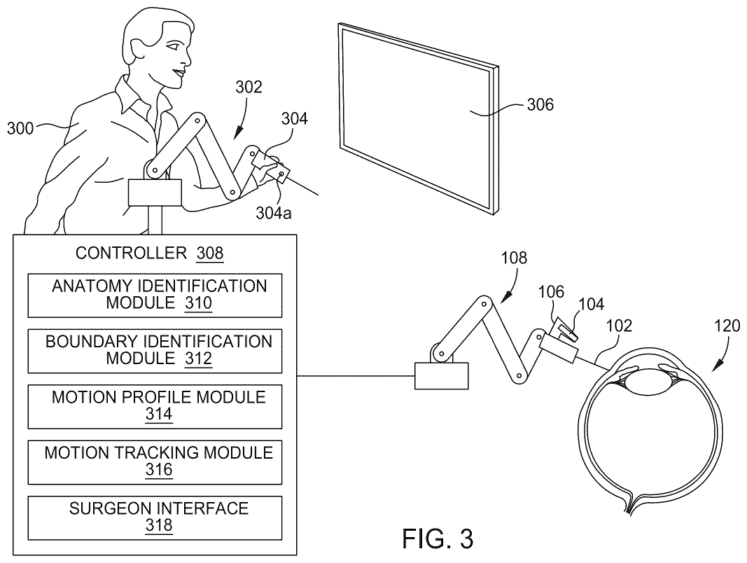

Referring to FIG. 3, the robotic arm 108 may be controlled by a surgeon 300 through various interfaces. In the illustrated embodiment, the surgeon 300 actuates a robotic arm 302. The robotic arm 302 may have the same number of degrees of freedom as the robotic arm 108 or may have more or fewer degrees of freedom. The robotic arm 302 may have the same configuration of links 112a-112f and joints 114a-114f or a different configuration. The robotic arm 302 may be the same size as the robotic arm 108 or may be scaled larger or smaller than the robotic arm 108.

The robotic arm 302 is one example of an interface for controlling the robotic arm 108. The robotic arm 302 may be implemented as any of the robotic positioning systems described above as possible substitutes for the robotic arm 108. The robotic arm 302 may also be replaced with a joystick, controller (e.g., a pad with buttons and one or more joysticks such as might be used to control a video game), foot pedal, or any other type of input device having sufficient degrees of freedom to control the degrees of freedom of the robotic arm 302.

In use, a surgeon may grasp a handle 304 at a distal end of the robotic arm 108, e.g., having a corresponding place in the joints and links of the robotic arm 302 corresponding to the end effector 106 of the robotic arm 108. The robotic arm 302 may include sensors, such as force torque sensors configured to sense the state of the joints of the robotic arm 302, which can then be processed to obtain the position and orientation of the handle 304. Additionally or alternatively, cameras, a local positioning system, ultrasonic position sensors, or any other type of position-detection approach may be used to detect the position and orientation of the handle 304 in three-dimensional space. The robotic arm 302 may further include actuators configured to control the position and orientation of the handle 304 and to provide a desired level of resistance to movement of the handle 304 as discussed in greater detail below.

Functions such as the opening and closing of forceps 218, extension and withdrawal of forceps 218, activating a treatment laser 210, 216, activating a pump, activating a phaco-vit tool, or other actions may be also be invoked using the robotic arm 302. For example, the handle 304 may include one or more interface elements 304a that, when interacted with by the surgeon 300, invokes any of the above referenced functionalities. In other embodiments, the robotic arm 302 includes one or more additional degrees of freedom such that movement in the one or more additional degrees of freedom invokes other functionality, such as opening and closing of forceps 218.

The surgeon may likewise be presented with a display device 306. The display device 306 may present the output of the accessory device 104 embodied as an image device, the output of a surgical microscope, or some other imaging device. The display device 306 may be used in a split-screen mode in which images of two or more different imaging modalities are displayed in different regions of the display device 306. Alternatively, an image according to one imaging modality may be displayed with an overlay that is an image according to another imaging modality or derived from an image according to another imaging modality, such as one or more labels for one or more items of anatomy. The display device 306 may be one of multiple displays each displaying images from a different imaging modality or different views according to the same imaging modality, such as different views or section planes of a three-dimensional image. The surgeon may therefore use the visual feedback on the display device 306 to guide movement of the handle 304. Movement of the handle 304 detected as discussed above may then be translated into corresponding movement of the end effector 106 of the robotic arm 108.

The sensors and actuators of the robotic arm 108 and the robotic arm 302 may be coupled to a common controller 308. The controller 308 may further receive images from some or all of the accessory device 104 embodied as an OCT, a surgical microscope, or other imaging device. The controller 308 may also receive images from an imaging device that is not mounted to the end effector 106 of the robotic arm 108, such as a surgical microscope having the eye 120 in the field of view thereof.

The controller 308 may include an anatomy identification module 310. The anatomy identification module 310 analyzes images received by the controller 308 and creates a three-dimensional image of the eye 120 and identifies anatomy in the eye, such as the cornea 122, lens 124, iris 126, retina 128, capsular bag, trabecular meshwork, Schlemm's canal, membranes on the retina, or any other item of anatomy constituting part of the eye 120. The anatomy identification module 310 may include some or more machine learning models, each being trained to label a particular item of anatomy. Each machine learning model may be embodied as a neural network, deep neural network (DNN), convolution neural network (CNN), recurrent neural network (RNN), Bayesian network, genetic algorithm, multiple linear regression model, multivariate polynomial regression model, support vector regression model, or any other type of machine learning model.

Inasmuch as images may be received throughout an ophthalmic treatment, the anatomy identification module 310 may continuously or periodically process these images to obtain a current labels of items of anatomy, which may reflect changes to the items of anatomy resulting from the ophthalmic treatment, such as the change in a membrane during a peeling process, the remaining vitreous during a vitrectomy, remaining portions of the lens 124 during phacoemulsification, incisions or stents placed in the trabecular meshwork, or other changes to anatomy of the eye 120.

The controller 308 may include a boundary identification module 312. The boundary identification module 312 may identify one or more items of anatomy, or a region adjacent an item of anatomy into which the distal end 200 of the instrument 102 should not enter, such as according to default settings or a treatment plan provided to the boundary identification module 312 for a given ophthalmic treatment. For example, for phacoemulsification, a boundary may include the capsular bag or an artificial surface offset inwardly from the capsular bag. For a vitrectomy, a boundary may include the retina, lens, and/or choroid or artificial surfaces offset inwardly from any of these items of anatomy. For a glaucoma treatment, the boundary may include the lens, ciliary body, or other items of anatomy that should not be contacted by the distal end 200 of the instrument 102. For a retinal peel the boundary may include any anatomy of the eye not covered by the membrane to be pealed, including portions of the retina over which the membrane does not extend. Other ophthalmic treatments may include boundaries corresponding to anatomy that should not be contacted during the procedure.

The controller 308 may include a motion profile module 314. The motion profile module 314 implements predefined movements using the robotic arm 108 or implements constraints on movements invoked by the surgeon 300 in accordance with one or more motion profiles. A motion profile may be defined with respect to items of anatomy such that the surgeon may select an item of anatomy, or a portion thereof, in order to invoke performance of one or more actions defined in the motion profile with respect to the item of anatomy or a portion thereof.

In a first example, the actions required to peel a membrane are very intricate and must not place undue pressure on the retina 128. Accordingly, a motion profile may include (a) a membrane grasping movement using forceps 218 at a location indicate by the surgeon that limits pressure on the retina 128 and/or grasps the membrane with an appropriate amount of clamping force or (b) limits imposed on a grasping movement invoked by the surgeon 300 that prevents undue pressure on the retina 128 and limits clamping force to the appropriate amount. The motion profile may by dynamically defined, such as with movements of the motion profile being calculated based on feedback from a force/torque sensor 220, such as to maintain outputs of the force/torque sensor 220 below a threshold value. In some embodiments, the threshold value is obtained by measuring force and torque values generated by a surgeon performing peeling manually. Additional feedback may be provided using the three-dimensional image. For example, if detachment of the retina begins to occur, pulling force may be decreased or stopped.

An artificial intelligence model may be used to control peeling. Peeling may be controlled by the artificial intelligence model with feedback and adjustment in response to feedback as described above, e.g., feedback from a force/torque sensor and/or feedback regarding retinal detachment.

The artificial intelligence model may be trained with a dataset in which each entry describes a membrane peeling of a region, whether a complete peeling of a membrane or a specific peeling movement. For example, each entry may represent an individual grasping and peeling movement. Each entry may record as desired outputs some or all of a pulling angle of the surgeon, a trajectory of pulling on the membrane, and values output by a force/torque sensor during peeling. Each entry may include, as inputs, a degree of adhesion of the membrane to be peeled and a portion of an OCT image representing the region peeled. Each entry may include a measure of patient outcome, such as whether and by how much retinal detachment occurred. The artificial intelligence model may be trained using the data entries to generate motion profiles, such as puling force, angle, trajectory, and a force/torque threshold for peeling retinal membranes without retinal detachment.

In some membrane peeling operations, a flap is raised using a scraper prior to grasping with forceps. A flap raising operation, including selection of the site for raising the flap, may likewise have a corresponding motion profile, which may likewise include an artificial intelligence machine learning model trained to perform this task. The motion profile may select such parameters as scraping force, angle, placement location, and pulling force. The flap raising motion profile may likewise be controlled according to feedback from a force/torque sensor. For example, pressing of the scraper against the membrane may be paused or slowed in response to output of the force/torque sensor exceeding a threshold value. Likewise, if separation of the retina is detected during raising of the flap, movements may be stopped or slowed. In some embodiments, a scraper may be used for peeling as well rather than forceps.

In some embodiments, one or more second machine learning models are used to automatically select a starting point for peeling, which may include an initial scraping step. The one or more second machine learning models may further generate a pattern for peeling an entire membrane. The one or more second machine learning models may take as inputs a three-dimensional image of a retina covered by a membrane, which may include a label identifying the portion of the three-dimensional image corresponding to the membrane. The one or more second machine learning models may be trained using data entries. Each data entry may include, as a desired output, a pattern followed by a surgeon removing the membrane and possibly a representation of a patient outcome, such as amount of retinal detachment, severity of retinal bleeding, or other metric. Each entry may include, as inputs, some or all of a three-dimensional image of the eye including the membrane, a label indicating the portion of the three-dimensional image corresponding to the membrane, a degree of adhesion of the membrane, or other values. The one or more second machine learning models may therefore be trained using the training data entry to generate a pattern for peeling a retinal membrane, including the starting and ending point of the pattern. For example, the artificial intelligence model may be trained identify areas of weak retinal attachment and perform peeling of such areas last, such as in a spiral pattern around the areas of relatively weak retinal attachment. Adjustments to the peeling pattern may be performed in response to feedback, e.g., e.g., avoid areas where retinal detachment occurs until a later point in the peeling pattern.

In a second example, the motion profile defines placement of a corneal suture or any other type of suture. Other suture substitutes such as staples or glue may be implemented in a like manner. For example, the motion profile may one or both of (a) define a series of movements of forceps 218 in order to place a suture at a location specified by the surgeon 300 and that places a proscribed amount of tension on portions of the cornea joined by the suture during keratoplasty (b) defines limits on movements of the forceps 218 invoked by the surgeon 300 in order to achieve a suture exerting the proscribed amount of tension. For example, for sutures placed around the perimeter of a transplanted cornea, it is desirable that the tension in all the sutures be equal to avoid deformation of the cornea. The motion profiles may therefore promote placement of sutures providing uniform tension. A motion profile for placing adhesive or using laser adhesion in place of sutures may also be defined.

In a third example, the motion profile defines placement of an incision or stent in the trabecular meshwork of the eye 120 or elsewhere in the eye in order to treat glaucoma. For example, the motion profile may one or both of (a) define a series of movements of the instrument 102 to place an incision or stent in a location selected by a surgeon or defined in a treatment plan (b) defines limits on movements of the instrument 102 invoked by the surgeon 300 in order to promote placement of an incision or stent to the correct depth and in a correct location within the anterior chamber of the eye 120.

In a fourth example, a surgeon 300 selects a point on an image to be treated by a treatment laser according to any of the embodiments disclosed above and invokes a motion profile to perform laser treatment (e.g., photocoagulation or retinal attachment). The point may be selected by tapping a touch screen, moving a cursor (e.g., reticle) with respect to the image using a pointing device, or some other input. In response to selection of the point, the controller 308 moves the distal end 200 of the instrument 102 to an appropriate distance and angle with respect to a location in the eye 120 corresponding to the point on the image and emits a pulse. The surgeon may also trace a line on an image to instruct the controller to emit a series of pulses at predefined intervals along a path along the anatomy of the eye 120 corresponding to the line. The treatment laser may be capable of emitting multiple independently guidable beams such that multiple points may be treated by the treatment laser simultaneously.

In a fifth example, a motion profile defines the grasping and extraction of a lenticle created as part of a SMILE procedure. For example, inasmuch as the lenticle itself may be created using a computer-controlled activation of a laser, the location of the lenticle may be known and a motion profile may then define extraction of the lenticle using forceps 218. The grasping and extraction may be performed with feedback from a force/toque sensor. The grasping and extraction may be performed by a machine learning model trained with data from previous grasping and extraction operations.

Other motion profiles may be used by the motion profile module 314 to perform or limit actions that are part of other ophthalmic treatments. The motion profiles used by the motion profile module 314 may be manually programmed. The motion profiles may be obtained by recording movements of an instrument 102 in a previous ophthalmic treatment, whether coupled to a handpiece held by the surgeon or controlled by way of the robotic arm 302.

The controller 308 may include a motion tracking module 316 configured to track movement of the instrument 102, particularly the distal end 200, relative to the anatomy of the eye. The motion tracking module 316 may identify representations of the instrument 102 in three-dimensional images generated during the ophthalmic treatment. The motion tracking module 316 may estimate a position and orientation of the instrument 102 in three dimensions and possibly first or second derivatives thereof. The motion tracking module 316 may predict a future location for the instrument 102, such as using Kalman filtering or another motion-prediction algorithm. For example, model-based estimation can predict one or more samples ahead and may be suitable for implementing the motion tracking module 316.

Accordingly, if the actual or predicted location of the instrument 102, particularly the distal end 200, is incident on or outside of a boundary defined by the boundary identification module 312, the controller 308 may prevent movement of the instrument 102 through the boundary. For example, prevention and/or resistance of movement of the instrument 102 through the boundary may be implemented according to the approaches described in the following documents, each of which is hereby incorporated by reference in its entirety:
U.S. Patent Application Ser. No. 63/315,129, filed Mar. 1, 2022, and entitled ROBOTIC IMAGING SYSTEM WITH FORCE-BASED COLLISION AVOIDANCE MODE; and
U.S. Patent Application Ser. No. 63/315,130, filed Mar. 1, 2022, and entitled ROBOTIC IMAGING SYSTEM WITH VELOCITY-BASED COLLISION AVOIDANCE MODE.

The controller 308 may include a surgeon interface 318. The surgeon interface 318 may receive inputs from the surgeon 300 in the form of voice commands, gestures, inputs to a touchscreen, pointing device, keyboard, joystick, foot pedal, or other input device. Surgeon 300 may provide inputs invoking performance or imposition of a motion profile, adjusting resistance of the robotic arm 302 to movement, activating the accessory device 104, adjusting the portion of the three-dimensional image displayed on the display device 306, adjusting the imaging modality whose images are displayed on the display device 306, or adjusting other aspect of the operation of the robotic arm 302, robotic arm 108, and accessory device 104.

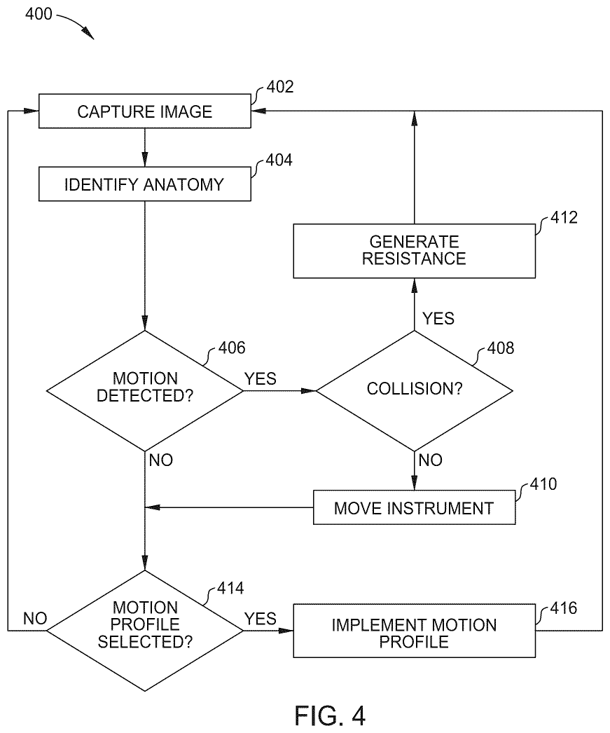

FIG. 4 illustrates an example method 400 that may be executed by the controller 308. The method 400 includes capturing, at step 402, an image. The image may be a three-dimensional image of the eye 120 captured using one or more imaging modalities as described above. The method 400 includes identifying, at step 404, anatomy of the eye 120 represented in the three-dimensional image, such as using any of the approaches described above with respect to the anatomy identification module 310.

The method 400 includes evaluating, at step 406, whether motion of the robotic arm 302 is detected. If so, the method 400 may include evaluating, at step 408, whether movement of the robotic arm 108 in correspondence with the movement of the robotic arm 302 would result in an actual or predicted collision of the instrument 102 with a boundary, such as a boundary identified by the boundary identification module 312 as described above and actual or predicted location of the instrument 102 being determined as described above with respect to the motion tracking module 316 described above.

If not, the method 400 may include moving, at step 410, the instrument 102 using the robotic arm 108 in correspondence with the detected movement of the robotic arm 108. If so, the method may include one or both of (a) refraining from moving the instrument 102 in correspondence with movement of the robotic arm 302 and (b) invoking actuators of the robotic arm 302 to generate, at step 412, resistance to movement of the robotic arm 302. For example, step 412 may include activating brakes in one or more joints of the robotic arm 302, activating actuators to generate torques opposing, but less than, torques exerted on the joints of the robotic arm 302 by the surgeon 300. In this manner, the surgeon is provided feedback to avoid making undesirable contact with items of anatomy labeled with boundaries.

In some embodiments, where a collision is only predicted, step 412 may include moving the robotic arm 108 in correspondence with movement of the robotic arm 302 with resistance to movement of the robotic arm 302 increasing with proximity of the instrument 102 to a boundary. In still other embodiments, the resistance to movement of the robotic arm 302 increases with proximity of the instrument 102 to a boundary starting at a predefined distance from the boundary, such as according to a linear, quadratic, or exponentially increasing function of proximity, e.g., A/x or A-x, where x is distance to the boundary and is a predefined parameter. In some embodiments, resistance to movement simulates a virtual spring interposed between the instrument 102 and a boundary and resisting movement toward the boundary and possibly recoil away from the boundary.

The method 400 may further include evaluating, at step 414, whether a motion profile has been selected by the surgeon, such as through the surgeon interface 318. If so, the motion profile is implemented at step 416, such as any of the motion profiles described above. Implementing the selected profile may include performing one or more predefined actions as defined in the motion profile at a location specified by the surgeon, such as the current location and orientation of the instrument 102. As noted above, implementing a motion profile may include imposing limits on actions invoked by the surgeon using the robotic arm 302 according to the motion profile. Feedback regarding limits imposed according to the motion profile may be provided in the form of resistance to movement of the robotic arm 302, such as movement of the robotic arm 302 away from a defined motion profile receiving greater resistance as described above.

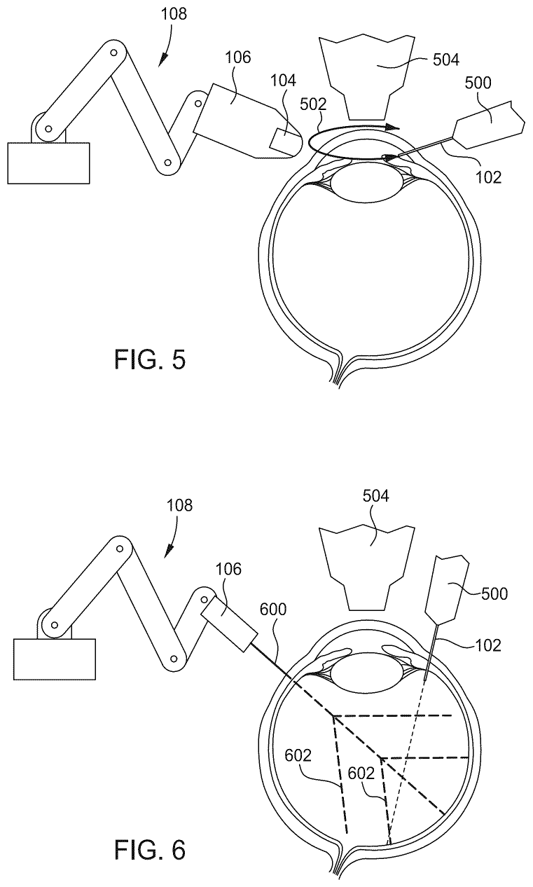

Referring to FIG. 5, in some embodiments an accessory device 104 is mounted to the end effector 106 of the robotic arm 108 whereas the instrument 102 is mounted to a handpiece 500 that is held in the hand of a surgeon or to a separate robotic arm. The accessory device 104 may be embodied as an OCT or other imaging device. In the illustrated embodiment, the accessory device may be moved to various points along a circular path 502 around the eye 120 and be oriented at various angles relative to the eye 120. The accessory device 104 may therefore be moved to positions and orientations that are (a) not currently obstructed by the instrument 102 and (b) position and orient the optical axis of the accessory device 104 at angles that are not substantially parallel and colinear to the optical axis of the eye 120, such as at angles of between 5 and 90 degrees relative to the optical axis of the eye and offset between 1 and 20 mm from the optical axis of the eye 120. This range of positions and orientations enables the accessory device 104 to image anatomy that might not be visible to a surgical microscope imaging the eye 120 through the iris 126 with the optical axis of the surgical microscope 504 substantially (e.g., within 5 degrees of) parallel to the optical axis of the eye 120.

The path followed by the end effector 106 may be controlled in various ways. In some embodiments, a treatment plan specifies movements of the end effector 106 throughout a procedure. The controller 308 may therefore move the end effector 106 in accordance with the treatment plan, such as in response to inputs from the surgeon indicating that a step in the treatment plan is completed or has begun. The movement of the end effector 106 may be automatic, such as in response to movement of the instrument 102. For example, the controller 308 may automatically position the end effector 106 such that the distal end of the instrument 102 is in the field of view of the accessory device 104 embodied as an OCT. In other embodiments, the position and orientation of the end effector 106 may be controlled by the surgeon, such as by means of one or more foot pedals, voice commands, hand gestures, or other inputs to the surgeon interface 318. For example, using a touch screen, the surgeon may specify a point and an orientation indicating that the OCT should be directed at that point with the optical axis of the OCT having the specified orientation. In another example, a surgeon may label areas of the eye 120, such as in a treatment plan. The surgeon may then select a labeled area in order to invoke imaging of that area using the OCT.

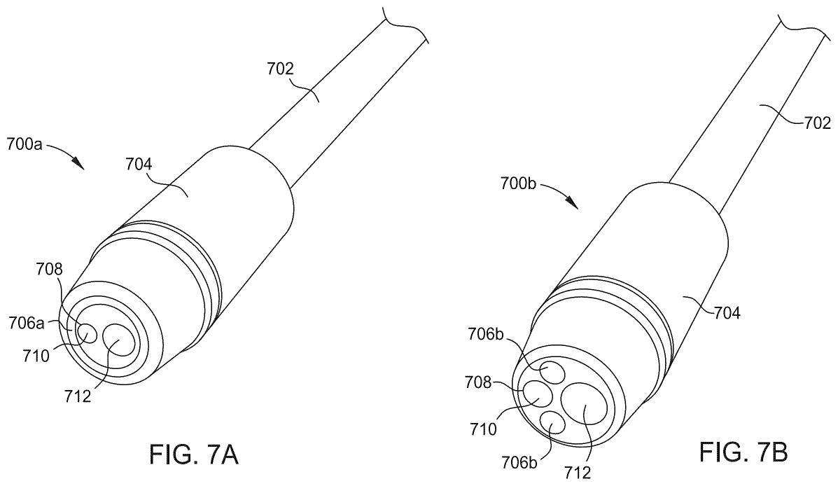

Referring to FIG. 6, in another implementation, the accessory device 104 includes a probe 600 adapted to insert within the eye 120. The robotic arm 108 may move the probe 600 through the same range of motion about an RCM located in an incision as described above with respect to the instrument 102. The probe 600 may include a fiber optic cable emitting light 602 into the posterior chamber or anterior chamber of the eye 120. For example, the probe 600 may be an endoscope with integrated light source as described below with respect to FIGS. 7A and 7B. The probe 600 may also be used to emit light onto the exterior of the eye 120.

The robotic arm 108 may be used to direct the light 602 onto a desired area of the eye 120. For example, in some embodiments, a surgeon 300 may select an item of anatomy or a region of the eye 120 from the three-dimensional image representing the eye 120, such as using the surgeon interface 318 as described above. The controller 308 may then select a position and orientation of the probe 600 in order to illuminate the item of anatomy or region. In particular, by withdrawing the probe 600 as shown in FIG. 6, the area of the eye 120 illuminated by the light 602 can be increased. For example, the surgeon 300 may trace a line on a touch screen in order to further insert or withdraw the probe 600 relative to an incision 130. In some embodiments, where the location of the instrument 102 is tracked, the controller 308 may select the position and orientation of the probe 600 to illuminate a region close to the distal end 200 of the instrument 102, e.g., within 5 mm, 3 mm, 2 mm, or 1 mm of the distal end 200.

Referring to FIGS. 7A and 7B, an instrument 102 and/or probe 600 may be embodied as the illustrated distal portions 700a or 700b. For example, a hollow rod 702 may include a fitting 704 secured thereto. A distal end of the fitting 704 may include a light source, such as the illustrated ring light source 706a for the distal portion 700a or one, two, or more individual light sources 706b for the distal end portion 700b. Each light source 706a, 706b may be embodied as one or more light emitting diodes (LED) either with or without a lens or diffuser for distributing light from the one or more LEDs. Each light source 706a, 706b may be a lens or other transparent structure that transmits light received from an optical fiber passing through the hollow rod 702.

The distal end of the fitting 704 may additionally define an opening 708. An objective lens 710 may be positioned within the opening. The objective lens 710 may focus light onto a camera positioned within the fitting 704 or onto an optical fiber conducting the light to a camera located elsewhere. For example, the objective lens 710 and camera may function as an endoscope. Images captured through the lens 701 may be presented on the display device 306, used to generate the three-dimensional image along with images from one or more other imaging devices, such as a surgical microscope and/or OCT, or for some other purpose.

The distal end of the fitting 704 may define an additional opening 712. The additional opening 712 may perform one or more functions. The additional opening 712 may permit a stent, infusion liquid, viscoelastic fluid, IOL, or other structure or fluid to be injected into the eye 120 of a patient. The additional opening 712 may be used to suction fluid from the eye 120. The additional opening 712 may be used to emit light from a treatment laser coupled to the additional opening 712 by an optical fiber or located within the fitting 704.

The fitting 704 may be visible within the eye 120 to an imaging device, such as the accessory device 104 embodied as an OCT. The fitting 704 may be visible due to the material from which the fitting 704 is formed or due to a marking material affixed thereto. The visibility of the fitting 704 facilitates identifying and localizing the fitting 704 in the three-dimensional image in order to perform motion tracking and enforce boundaries as described above. The fitting may be removably attached to the hollow rod 702 and may be disposable.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A system for performing ophthalmic surgery, the system comprising:
a robotic positioning system including an end effector, the robotic positioning system configured to position the end effector with at least five degrees of freedom;
an ophthalmic surgical instrument mounted to the end effector; and
an accessory device mounted to the end effector and configured to facilitate performance of an ophthalmic treatment by the ophthalmic surgical instrument.

2. The system of claim 1, wherein the accessory device is a treatment laser.

3. The system of claim 1, wherein the ophthalmic surgical instrument is a phaco-vit instrument.

4. The system of claim 1, wherein the ophthalmic surgical instrument is configured to emit treatment laser pulses.

5. The system of claim 1, wherein the ophthalmic surgical instrument comprises an opening for injecting at least one of a structure and a fluid into an eye of a patient.

6. The system of claim 1, wherein the ophthalmic surgical instrument comprises forceps.

7. The system of claim 6, wherein the ophthalmic surgical instrument comprises a sensor configured to sense at least one of force or torque transmitted through the forceps.

8. The system of claim 1, wherein the ophthalmic surgical instrument is an endoscope.

9. The system of claim 1, wherein the ophthalmic surgical instrument comprises a light emitting structure.

10. The system of claim 1, wherein the accessory device is an imaging device.

11. The system of claim 10, wherein the imaging device is an optical coherence tomography (OCT) imaging device.

12. The system of claim 11, further comprising, a controller coupled to the OCT imaging device, the controller configured to generate a three-dimensional image of an eye of a patient according to images received from the OCT imaging device.

13. The system of claim 12, wherein the controller is further configured to:
define a boundary with respect to anatomy of the eye represented in the three-dimensional image;
detect a representation of the ophthalmic surgical instrument in the three-dimensional image; and
restrict movement of the ophthalmic surgical instrument through the boundary.

14. The system of claim 13, wherein the robotic positioning system is a first robotic positioning system, the system further comprising a second robotic positioning system and a handle secured to the second robotic positioning system;
wherein the controller is further configured to:
control the first robotic positioning system to position the end effector in correspondence with a position and orientation of the handle; and
restrict movement of the ophthalmic surgical instrument through the boundary by resisting movement of the second robotic positioning system.

15. The system of claim 12, wherein the controller is further configured to:
receive invocation of a motion profile; and
execute the motion profile with respect to an eye of the patient.

16. The system of claim 15, wherein the motion profile defines actions implementing placement of a suture in an eye of a patient.

17. The system of claim 15, wherein the motion profile defines actions implementing peeling of a membrane from a retina of a patient.

18. The system of claim 15, wherein the motion profile defines actions implementing placing at least one of an incision or a stent in a trabecular meshwork of a patient.

19. A system for performing ophthalmic surgery, the system comprising:
a robotic positioning system including an end effector, the robotic positioning system configured to position the end effector with at least five degrees of freedom;
an imaging device mounted to the end effector;
a controller coupled to the robotic positioning system and the imaging device, the controller configured to:
control the robotic positioning system during performance of an ophthalmic treatment with respect to an eye of a patient to change a position and orientation of the imaging device with respect to the eye of the patient to capture section images of the eye of the patient;
generate a three-dimensional image of the eye of the patient during performance of the ophthalmic treatment; and
output at least a portion of the three-dimensional image to a display device during performance of the ophthalmic treatment.

20. The system of claim 19, wherein the controller is configured to select the position and orientation of the imaging device with respect to the eye according to a treatment plan for the ophthalmic treatment.
